(19) United States
(12) Patent Application Publication
(10) Pub. No.: US 2004/0068162 A1
(43) Pub. Date: Apr. 8, 2004

(54) Apparatus and method of personal screening for cervical cancer conditions in vivo

(76) Inventor: Vaclav Kirsner, Bellvue, CO (US)

Correspondence Address: VACLAV KIRSNER, 83 DAVIS RANCH ROAD, BELLVUE, CO, 80512, US

(21) Appl. No.: 10/609,795

(22) Filed: Jun. 30, 2003

Related U.S. Application Data
(60) Provisional application No. 60/392,552, filed on Jul. 1, 2002.

(51) Int. Cl. A61B 5/00

(52) U.S. Cl. 600/300

(57) ABSTRACT

A method and apparatus for personal screening for early signs of cervical cancer is claimed, whereby the user performs daily or almost daily a diagnostic self-check for some other aspect of reproductive health, and the electronic testing for cervical cancer type of tissue aberration is performed automatically in the background. The screening is invisible to the user, causing no anxiety and no discomfort. The user only becomes alerted to the need to see a physician if a preset condition of reproducibility is reached in the background evaluation of the measurement data if the aberrant pattern has been detected consecutively in a preset number of menstrual cycles, the device prompts the woman to see a physician with a view to undergoing a more demanding definitive diagnostic examination such as colposcopy with biopsy. The invention provides the diagnostic screen in a manner that does not cause the discomfort, anguish and anxiety associated with the Pap smear screen of the prior art.

### FIELD OF THE INVENTION

This application discloses an electronic device and system that monitors the physiological status of epithelial tissues in the posterior fornix and/or cervical region of the vagina. The invention relates to a method and apparatus for women's self-administered reproductive health monitoring, with a means to evaluate the data and issue a prompt when early signs of cervical cancer or pre-cancerous development are detected, and to forward the data to the health care system or an individual health provider. More particularly, this invention relates to an in vivo probe for personal use enabling the early detection of cervical cancer or of pre-cancerous state and cervical dysplasia. The invention provides an electronic system for evaluating and communicating the data, providing the diagnostic screen in a manner that does not cause the anguish and anxiety associated with the Pap smear screen of the prior art.

### BACKGROUND AND PRIOR ART

Cervical cancer, or carcinoma of the cervix uteri, is one of the most common malignancies in women worldwide (about 25% of all female cancers). Although it accounts for only about 2.5% of all cancers diagnosed in American women today, it was the most common cause of cancer deaths in American women in the 1930s. With the introduction of the Pap smear, early detection and treatment of pre-invasive disease became possible. Although the incidence of cervical cancer has fallen dramatically in the US, the trend has reversed since the mid-1990s. Approximately 50 million Pap smears are performed each year in the United States. This number is to be seen in the context of the total population of the U.S. (287,180,787), and of the world (6,228,245,253 as of Jun. 2, 2002), where women constitute approximately one half of the numbers.

These statistics mean that at most approximately 35% of American women undergo the Pap smear screening (this is an over-estimated percentage since some of the smears are repeats). The increased use of the Pap smear is largely responsible for a significant reduction in the number of deaths from cervical cancer in the U.S. over the years (e.g., a decrease of. 74% from 1955 to 1992), consistent with the fact that the cancer can be controlled. Worldwide, however, cervical cancer is second only to breast cancer in both incidence and mortality. In many developing countries, where preventative health measures are not routine, the incidence and mortality from cervical cancer continue to rise, because of the low use of the so-called Pap smear screening program. [“Pap” after George. Papanicolaou (1883-1962), an American anatomist.].

In view of the 35,000 obstetricians and gynecologists in the U.S. (as of August 2000), the above statistics also mean that, on average, approximately 5 Pap smears are performed per American obgyn [obstetrician and gynecologist] per working day. This may be seen in the context of the ratio of approximately 4,100 women to every US obgyn (287,180,787/2/35,000), which suggests that, for a complete screening coverage, 16 women would have to be seen for a Pap smear by every US obgyn every day. (260 working day per year, 52 weeks, 5 working days per week). This required daily average goes up if the number of days goes down (such as 21 women per day per obgyn at 200 Pap smear days per year).

Many factors have been implicated in the high incidence of cervical cancer and of the underlying problem of abnormal cell growth in the cervix (dysplasia) that sometimes progresses to cervical cancer. The risk factors include the following: 1. having sex before the age of 18; 2. having had sex with many partners; 3 having been infected with HPV (Human Papilloma Virus) or with some other sexually transmitted disease (STD); 4. having had sex partners infected with STDs or HIV/AIDS; 5. age over 60; 6. smoking; 7. having had several pregnancies; 7. being more than 30 lbs overweight; 8. taking birth control pills, 9. poor nutrition; and 10. family history of cervical cancer. These factors tend to be on the rise. Dysplasia is most likely to occur in women between the ages 25-35, many years before the cancer may eventually strike.

Cervical dysplasia is a laboratory diagnosis. It is made on a, sample of cervical tissue taken by a gynecologist for a Pap smear analysis, which is performed later by a cytopathologist. [Depending on the specific naming system used, cervical dysplasia may also be called squamous intraepithelial lesion (SIL) or cervical intraepithelial neoplasia (CIN).]HPV is the sexually transmitted virus that causes 99.7 percent of all cases of cervical cancer. However, the presence of HPV does not necessarily indicate cancer. Ninety percent of cervical cancers initiate in the surface cells that line the cervix. When cancer cells multiply and spread to surrounding tissues, the bloodstream or lymphatic system can become infected. It takes months and even years for cervical cancer to become invasive cancer. As a consequence, invasive cancer appears mostly in women between the ages of 40-60. In USA, about 60 million women are aged 35-69, the population in which invasive cervical cancer appears the most and therefore most threatened by the disease (fewer than 5% of patients who develop recurrent carcinoma of the cervix are alive five years later).

Dysplasia is a pre-malignant condition. The next stage, noninvasive cancer, occurs when dysplasia progresses to the outer layer of the cervix. If the cancer is untreated in the noninvasive phase, it eventually spreads to deeper layers of tissue and then onto other organs, such as the uterus, rectum, and bladder.

In its early stages, cervical cancer has no detectable symptoms.

Contrary to the overall falling trend in the U.S., the incidence of cervical cancer adenocarcinoma has more than doubled between the early 1970s and the mid-1980s among women younger than 35 years of age, and currently accounts for more than 10% of all cervical cancers. It appears that there is a trend towards an increase in cervical cancer in younger women, which may account for the recent reversal in declining cervical cancer rates in the US. The cause of this trend is not known but the use of oral contraceptives (most prevalent among the youngest women) has been suspected as a contributing factor, related to higher frequency of promiscuous sexual activity and consequent infections.

This may become an effective motivating factor for young women to use a new approach to health monitoring. According to a study with racially diverse females of 13 to 22 years of age, benign cervical lesions caused by the (sexually transmitted) human papillomavirus (HPV) nearly always go away on their own in teens and young women (in 95%). These are cervical abnormalities—called low-grade squamous intraepithelial lesions (LSIL), and the current recommendation is that young women with LSIL get Pap smears every 6 months, but that physicians wait at least 18 to 24 months before considering treatment. The concern is that treatment is inappropriate in adolescents because of the costs and, significantly, the potential that the repeated cervical procedures could leave scar tissue and lead to later infertility (which is already on the rise).

There is also another potentially effective motivating factor: for young women to use a new approach to health monitoring. This should come from the increasing health-awareness and the interest in disease prevention—in the era of “sexual freedom”. One such aspect is the implication of HPV as the sexually transmitted factor causing the development of squamous-cell carcinoma of the cervix. The other aspect is that, whereas penile warts in males only very rarely result in cancer of the penis, when transmitted to the cervix, cancer is much more likely to follow. About one-third of patients who have a confirmed HPV infection of the cervix can be expected to develop cervical intraepithelial neoplasia (CIN) within a year. The unique characteristics of the cervix, coupled with other factors such as smoking, contribute to the onset of the cancer. Treatment of women with precancerous lesions involves surgical extirpation (removal, extermination) of the affected area.

While cervical cancer is curable if caught early, it is a fact that, overall, fewer than 5% of patients who develop recurrent carcinoma of the cervix are alive five years later.

The main problem with the existing Pap smear diagnostic system is as follows: The deadly harm caused by false negatives, and the anguish and expense caused by false positives, taken together, are the main problem with the existing Pap screening system. The major failure of the Pap smear system is the inability to persuade all women to undergo regular check ups.

In the U.S., an estimated 15,700 women per year were diagnosed with invasive cervical carcinoma in the mid-1990s (which means approx. 0.01% of all US women, and less than half a diagnosis per US obgyn). Although the cancer is eminently curable if diagnosed early, an estimated 4,900 women per year died of cervical cancer in the mid-1990s (31% of diagnosed). With false-negative rates of up to 40 percent in Pap smears from women later found to have cervical cancer (adenocarcinoma in situ and/or invasive endocervical adenocarcinoma), the inherently high rate of false negatives is the major problem in the existing system of screening. An increasingly prevalent problem is the occurrence of invasive cancer within 3 years of a negative Pap smear.

Regarding the false positives, every year more than 2 million American women enter “medical purgatory” when their Pap smears come back inconclusive because the laboratory finds something abnormal but cannot tell if it is an early sign of cervical cancer or not. According to specialist sources, approximately 2% of routine cervical smears show abnormal cytology, indicating an epidemic. These women receive the diagnosis of “ASCUS”—their Pap smears contain “Atypical Squamous Cells of Undetermined Significance” or “Atypical Glandular cells of Undetermined Significance” (“AGUS”)—but there remains poor agreement among cytopathologists in reclassifying the ASCUS/AGUS lesions, and in separating cancerous from benign lesions. For even a minor infection can cause an abnormal Pap smear. This means that the Pap has poor diagnostic specificity.

Many physicians recommend simply repeating the Pap smear. But at least three repeat tests must be done, one every four to six months, to be sure that no danger signs were missed. Therefore, it can take over a year to be sure that an ASCUS result was a false alarm. Also, missed work, childcare issues and the price of the visits to the doctor may make this procedure a hard choice for some women.

An alternative is colposcopy, in which doctors examine the vagina and cervix with a lighted magnifying, instrument and, usually, take a biopsy (a snip of cervical tissue to be examined under the, microscope). Results from a colposcopy with biopsy are usually definitive and obtained quite quickly—but the biopsy can be painful and it costs about $450. This diagnostic situation has posed a particular dilemma in clinical management decisions, both from the cost-benefit standpoint and the desirability of not subjecting patients to unnecessary invasive procedures.

The diagnostic errors in the Pap smear screening have been attributed in part to technical problems, but certain studies have also indicated that the errors may be primarily due to human factors that are not remedied by rules and regulations. Because human error may be a major contributor to false-negative readings of Pap smears, computer-assisted image analysis and artificial intelligence have been introduced as a means of improving the reliability of the Pap smear screening. For example, the PapNet, AutoPap, and Thin-Prep innovations are refinements to the time-honored Pap smear technique, while HPV typing is believed to offer a discrete adjunct to the Pap screening system, and is to be incorporated with certain new sampling techniques. Practitioners of the Pap screen methodologies believe that these technologies (HPV typing and computerized re-screening) could be incorporated into an efficient and logical triage system, and that this kind of pre-colposcopy triage (i.e., sorting patients for colposcopy) could potentially reduce the number of false-negative Pap smears, as well as-the number of false-positive cases referred for colposcopy.

Attempts to reduce laboratory error in the processing of Pap smears have encouraged the automation of cytology, with developments on three fronts. Firstly, the, use of liquid-based smear preparation (which prepares a slide in the laboratory); secondly, the use of microscope tracking to ensure that the cytotechnologist examines all areas of a slide; and thirdly, computer-assisted image analysis to re-screen conventionally prepared smears. All procedures ultimately rely on conventional cytopathology review, and “they still do not detect all abnormal cases” [Statement on technical devices for innovation in cervical cytology screening editorial. Am J Clin Pathol 1996; 106: 441.] Some of these techniques have been heavily marketed, both directly to women and to general practitioners.

However, the cost-effectiveness of this approach to screening the general population has not been established. In fact, mathematical models showed that manual methods of re-screening provide superior cost-benefit ratios when compared with automated approaches to Pap screening, and “the incremental benefit of automated cytology over conventional cytology is really quite small, and is unlikely to be substantially better than having a repeat Pap smear two years later” (The Medical Journal of Australia 1997; 167: 460-461).

Consequently, despite the advances in the Pap smear methodology, the situation continues and every year many women “enter medical purgatory” when their Pap smears come back inconclusive because the laboratory finds something abnormal but cannot tell if it is an early sign of cervical cancer or not. Therefore, to recap, because of the Pap's poor diagnostic specificity, the deadly harm caused by false negatives and the anguish and expense caused by false positives are the main problems with the existing Pap screening system.

Another problem with the existing screening system is that many women may not undergo the quite frequently needed invasive procedure either at all or regularly enough. This is because the speculum examination, involved in the Pap procedure (as illustrated in FIG. 1), is uncomfortable and embarrassing for some or many women; it requires direct viewing into the vaginal cavity, and as such must be performed by a physician or nurse practitioner, thus also adding to the expense and inconvenience. While cervical cancer is curable, because of its slow rate of growth, it must be caught early enough in order to respond to the cure. The present invention solves this problem by providing a personal reproductive health tool that performs the screen unobtrusively in the background.

Another problem with the existing screening system is that it involves a costly and inconvenient, invasive and anxiety-causing, procedure. Yet, according to certain medical sources, only 5 to 10 percent of the inconclusive Pap smears turn out to be cancerous or pre-cancerous. There is a great need for a screening procedure that sorts out which women really are at risk, avoiding unnecessary anxiety in the rest. This need is the problem that the present invention aims to solve—by providing a personal women's health tool, ostensibly for such a non-threatening purpose as fertility monitoring, whereby the pathological warning is incidental to the main purpose of the product. Fertility monitoring is inherently of great interest among reproductive-age women, including those aged 25 to 35, the age group that is at the greatest risk of the underlying problem of abnormal cell growth in the cervix (dysplasia) that sometimes progresses to cervical cancer. This is also the age group, in which the problems of inability to conceive when desired begin to emerge.

An example of a recent patent in the area of improvements to the Pap smear screening methodology is U.S. Pat. No. 6,379,907 “Diagnostic method using expression of MN/CA9 protein in AGUS Pap smears” assigned to the Regents of the. University of California (Oakland, Calif.). This prior art recognizes a, benign condition in a Pap smear when MN/CA9 antigen is, absent from both a typical cells and normal endocervical cells. An example of a different approach to cancer diagnosis, not necessarily within the Pap smear approach to the screening of the female population, is U.S. Pat. No. 6,391,554 “Detecting cancerous conditions by assaying for telomerase activity”. This prior art patent is assigned to Geron Corporation (Menlo Park, Calif.) and Board of Regents of the University of Texas Systems (Austin, Tex.), and it allows cancer to be detected even in tissues and cells which are not positive by pathology.

An example of a recent patent application in the cervical cancer diagnosis area is U.S. Patent Application 20020049441, “Surgical biopsy instrument”, Ser. No. 09/935,877, filed from the UK.

An example of a recently issued patent for a vaginal probe and its use is U.S. Pat. No. 6,080,118 “Vaginal probe and method of using same”, which claims a configuration suitable for multi-sensor analysis of body fluids directed towards the plurality of axially spaced sensors upon insertion in the user's mouth, anus or vagina, whereby body fluids collect in a channel ensuring that the sensors therein are emersed in the fluid and ensuring more accurate biosensor readings. The monitored body characteristics include body temperature, pH, LH level, mucous density, vaginal cavity pressure, estrogen level, progesterone level, and HCG level. Sensors for sensing covalent immobilization of haptens, polypeptides, enzymes, antibodies and DNA fragments are also claimed, as is an ultrasonic sensor at the tip of the vaginal probe.

Another example of a recently issued patent that discloses improvements in a diagnostic system for determining and/or monitoring a physiological condition in a female mammal is U.S. Pat. No. 6,364,844 “Diagnostic system for determining and/or monitoring physiological conditions in mammals”. The disclosed system utilizes salivary and vaginal mucus electrical resistance data measured via an oral and a vaginal electrical resistance probes, as input for improved “algorithms being capable of determining, substantially automatically, variables that can be used to define the beginning of the fertile cycle of the mammal, the end of the fertile cycle of the mammal and the most fertile day of the mammal.”

Neither of these examples of patented prior art solves the main problems in cervical cancer screening, reviewed above. The major failure of the Pap smear screening remains the inability to persuade all women to undergo regular check ups.

### SUMMARY OF THIS PATENT APPLICATION

In accordance with the inventions of the present application, a probe is provided which, in conjunction with an electronic module, provides a new screen for early detection of cervical cancer. The apparatus of this application measures in the vagina a physical parameter sensitive to the physiological status of the epithelial tissues. The probe is of such dimensions and shape as to place the active elements in contact with a well-defined predetermined aspect of the epithelial tissues in the vagina.

One object of the inventions of this application is to provide apparatus and method of screening that improves the acceptability, sensitivity and selectivity over those of prior art for detecting abnormalities in the epithelial tissues in the cervical region of the vagina, such as particularly the Pap smear methodologies.

Another object of the inventions of the present application is to increase the reliability and effectiveness of diagnostic screening for cervical cancer.

Another object of the inventions is to provide a diagnostic screen that avoids causing the woman user anguish and anxiety over the outcome of the diagnostic procedure, which is a major problem in the prior art methodologies. The present invention achieves that because the cancer screen is carried out in the background while performing fertility monitoring, which is inherently of great interest among women, including those aged 25 to 35 who are at the greatest risk of the underlying problem of abnormal cell growth in the cervix (dysplasia), which sometimes progresses to cervical cancer.

Another object of the inventions of this application is to provide a diagnostic screen on which to base the healthcare decision whether or not to undergo the costly and painful—but definitive—procedure of colposcopy with biopsy.

Another object of the inventions of this application is to monitor a parameter characteristic of the physiological and endocrinological status of the epithelial tissues. By doing this for the sake of guiding the management of therapy such as hormone replacement therapy in peri-menopausal women, i.e., in women between the ages of 40-60 where invasive cervical cancer appears the most, the present application aims, to solve the problem that in its early stages, cervical cancer has no detectable symptoms.

Another object of the inventions of the present application is to provide a diagnostic screen that inherently generates data frequently, regularly, and over the whole life span of the woman user. As an example of such a screen, fertility monitoring is inherently of great interest among reproductive-age women and (because apparent sub-fertility problems tend to emerge in these years) particularly those aged 25 to 35 who are at the greatest risk of the underlying problem of abnormal cell growth in the cervix (dysplasia), which sometimes progresses to cervical cancer. Even younger women and adolescents may be motivated to use this self-monitoring approach to reproductive health. Similarly, concerns about proper management of hormone replacement therapy in the peri-menopausal years motivate these women to use the apparatus and methods of the present invention, and in so doing provide for early cancer detection when other symptoms are not detected.

Another object of the present inventions is to provide self-monitoring means for women pursuing health awareness, primarily or ostensibly for fertility awareness, and only somewhat incidentally providing an appropriate warning if a consistently aberrant response is detected, thus avoiding the unhealthy state of anguish, uncertainty and anxiety, which is strongly associated with the Pap smear procedures of prior art. Fertility monitoring is inherently of great interest among women aged 25 to 35 who are at the greatest risk of the underlying problem of abnormal cell growth in the cervix (dysplasia) that sometimes progresses to cervical cancer.

Another object of the inventions of this application is to provide a diagnostic tool for physicians and the health care system, to more effectively manage their female patients. In terms of cervical cancer prevention, the object of the inventions of this application is a significantly greater outreach to the female population than the approximately 5 Pap smears per day per gynecologist in the U.S. today, and even fewer in many other countries. In terms of prevention, the objective is to facilitate a significant improvement on the statistic of about 1.4 cervical cancer deaths per 10 American obgyns (or 7 obgyns for each death). The first priority should be to screen more women, and the present application aims to facilitate meeting that objective.

### BRIEF OVERVIEW OF THE PREFERRED EMBODIMENTS

The purpose of the invention is to detect a tissue condition in the vagina, indicative of an early stage of the development of cervical. cancer, or of pre-cancerous state that warrants further investigation such as by colposcopy with biopsy, which detection will preferrably be incidental to the primary purpose of detecting the fertility status or hormonal status of the tissues.

The two main components of the invention include (1) a vaginally inserted detector probe, and (2) an electronic module for performing measurements with the probe's active elements and for evaluating, and communicating the measurement results.

Name and description of the structure of main component 1.

Component name: Probe.

Structure of main component 1: The probe is comprised of an electronic biophysical or electrochemical detector device capable of contacting tissues in a predetermined location within the vagina. It is made of a medical grade plastic in such a manner as, to have a smooth surface that is biocompatible and is not prone to allow adherance of microorganisms. One example of such material is the silastic made by Corning, and another is food-grade nylon.

Name and description of the structure of main component 2.

Component name: Electronic module.

Structure of main component 2: The electronic module is comprised of a structure that contains the electronics required for the measurement, evaluation of data, and for communicating the measurement result, and for communicating the stored raw data upon demand.

The inventions of this application comprise an improved method for detecting the aberrant pre-cancerous or cancerous state of the cervix by simple and routine self-monitoring of fertility and/or other aspect of reproductive health by individual women, and a method of monitoring the hormonal and physiological status of the epithelia in a defined region of the vagina (collectively referred to as “tissue bioassay”). In either case, the method involves the following steps:

placing in the vagina a probe having one or more active elements. Examples of such active elements: an electrode, or a light emitting diode, or a radio frequency emitter, or a high frequency ultrasound emitter, or emitter of any other form of energy which would be attenuated based upon the condition of the epithelial tissue,

measuring with the active elements at least one physical parameter indicative of the phase of the menstrual cycle or of the tissue's hormonal or physiological status, respectively and/or simultaneously (that is, doing both), and

comparing the value with a reference value or values whereby evaluating the diagnostic meaning of the said value, and

displaying the result of the evaluation on the dispay, and

optionally downloading substantially all stored raw data upon demand.

Preferred embodiments of the probe and electronic module components of the apparatus for this method include the following:

a—means for introducing the active elements into the vagina;

b—means for the measurement with the active elements of a physical variable under the control of the electronic module;

c—means for the measurement with the active elements of a physical variable characteristic of the hormonal and physiological variations in the tissues. Examples are electrode current, admittance or any of their components, or light or radio waves or ultrasound, or-other, derived or computed, parameter or any kind of electromagnetic energy, using for the active elements' stimulation any of a number of conceivable waveforms such as but not limited to at least one sine-wave;

d—means for the evaluation of the instant measurement data;

e—means for the communication of the evaluation of the instant measurement results;

f—means for downloading the stored raw data upon demand.

### BRIEF DESCRIPTION OF THE DRAWINGS

FIG. 1 is a schematic representation of the human female's circumstances within the methodology of the Pap smear prior art, shown in three parts, 1A through 1C, and indicating why women tend to dislike the experience.

FIG. 2 is a perspective view of one preferred embodiment of the apparatus of the present invention.

FIG. 3 is in two parts, 3A and 3B, and represents the probe of FIG. 2 inserted in the fornix of the vagina, with two different orientations of the active elements of the probe.

FIG. 4 is a block diagram of the second main component referred to above as the electronic module.

FIG. 5 shows that the telemetric embodiment 50 of the present invention includes a transmitting vaginal insert 51, receiver 52, and microprocessor system 53.

FIG. 6 shows that the housing 54 of the transmitting vaginal insert embodiment of the probe component may have any of a number of physical shapes such as a capsule (bolus) or a ring or a cylinder.

FIG. 7 illustrates the insertion of one type of the vaginal insert embodiments of the probe, namely the ring.

### DETAILED DESCRIPTION OF THE PREFERRED EMBODIMENTS

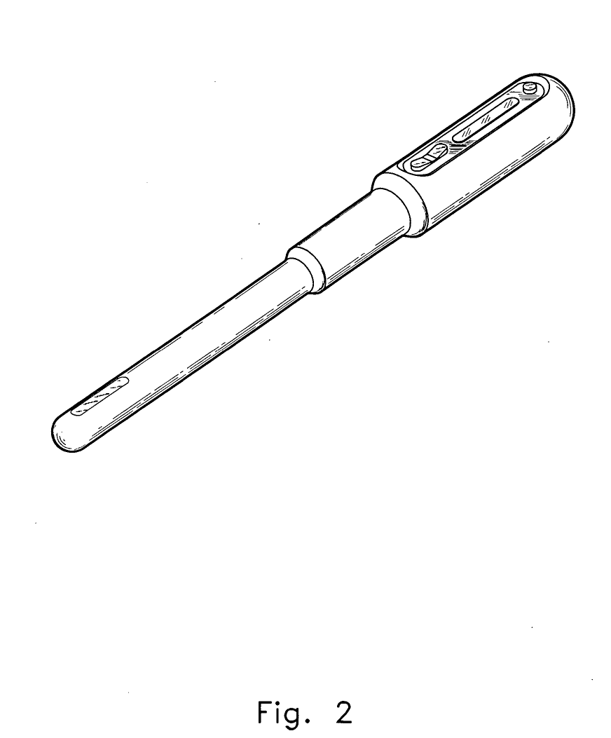

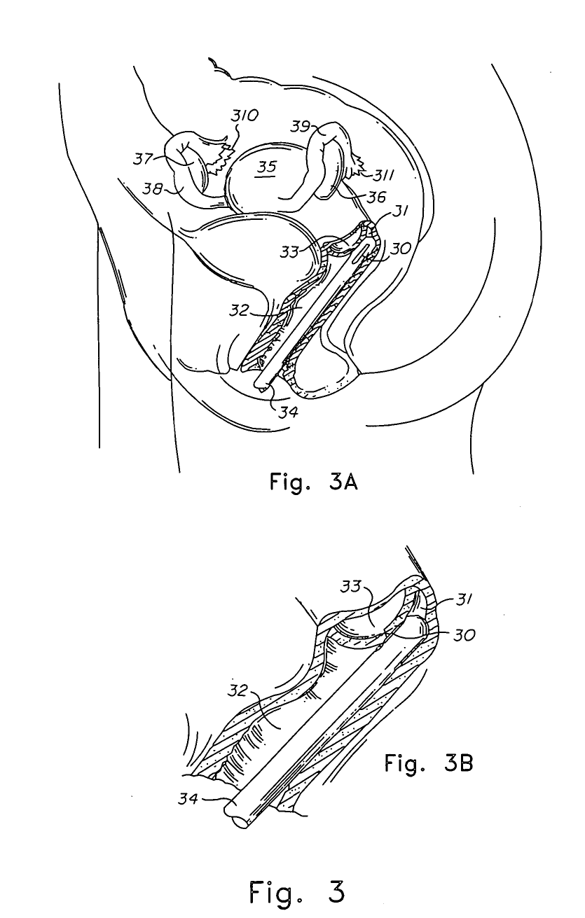

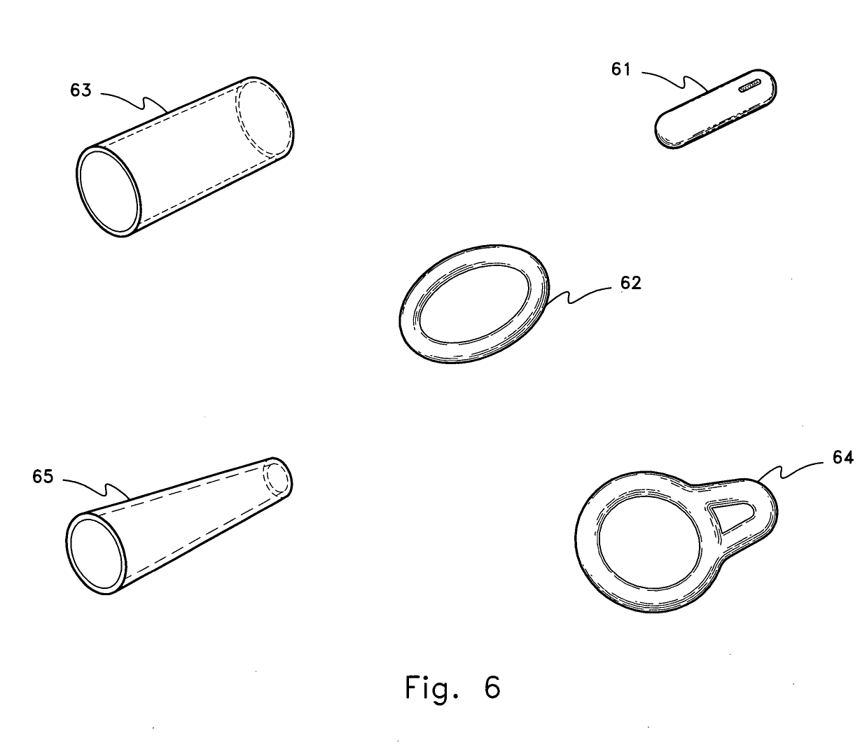

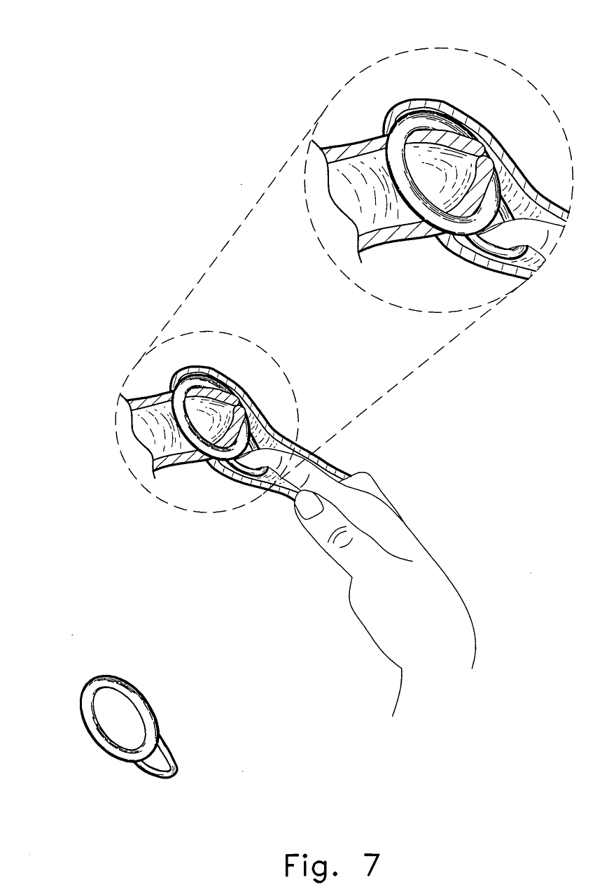

The probe component is comprised of an electronic detector device capable of contacting tissues in a predetermined location within the vagina. This placement is accomplished in one of two ways, illustrated in FIGS. 3 and 7, depending on whether the probe is physically connected with the electronic module or whether the probe is not permanently and physically joined with the electronic module, making merely an occasional electronic communication, contact such as via electromagnetic waves. These two alternative embodiments are respectively referred to as the main single-piece embodiment, and as the alternative telemetric embodiment of the probe. The placement of the main single-piece embodiment of the probe is accomplished simply by a daily or almost daily insertion as if a sanitary tampon were being inserted, as illustrated in FIG. 3. The placement of the alternative telemetric embodiment may be accomplished in a manner similar to inserting a cervical cap or a contraceptive diaphragm. The housing structure preferably is small in size to fit the size of the human vagina. The main, single-piece embodiment of the probe has an elongated structure as shown in FIG. 2, whereas the the structure of the transmitting vaginal insert may have any of a number of conceivable shapes, some of which are illustrated in FIG. 6, merely to indicate that there are numerous solutions.

The electronic module component is comprised of the microelectronic circuitry required to perform the following steps,

1. stimulate the active element(s) whereby performing the instant measurement;

2. store the instant measurement data in memory;

3. evaluate the instant data in terms of the primary purpose such as fertility status determination, by applying a program that essentially looks for a fit of the instant data point into a standard pattern; and/or

4. evaluate the instant data in terms of the secondary purpose, by applying another data examination program; and

5. display the outcome of step 3 or of step 4 on the digital display of the electronic module. An additional part of the electronic Module, generally a, port, allows the module to optionally perform the last main step; namely,

6. communicate the outcome of the instant diagnostic measurement, and optionally all stored raw data, to an external device for examination by a specialist. The communication is by any of a conceivable electronic means such as are involved in corded or cordless telephony and conceivably communicate the data over the Internet.

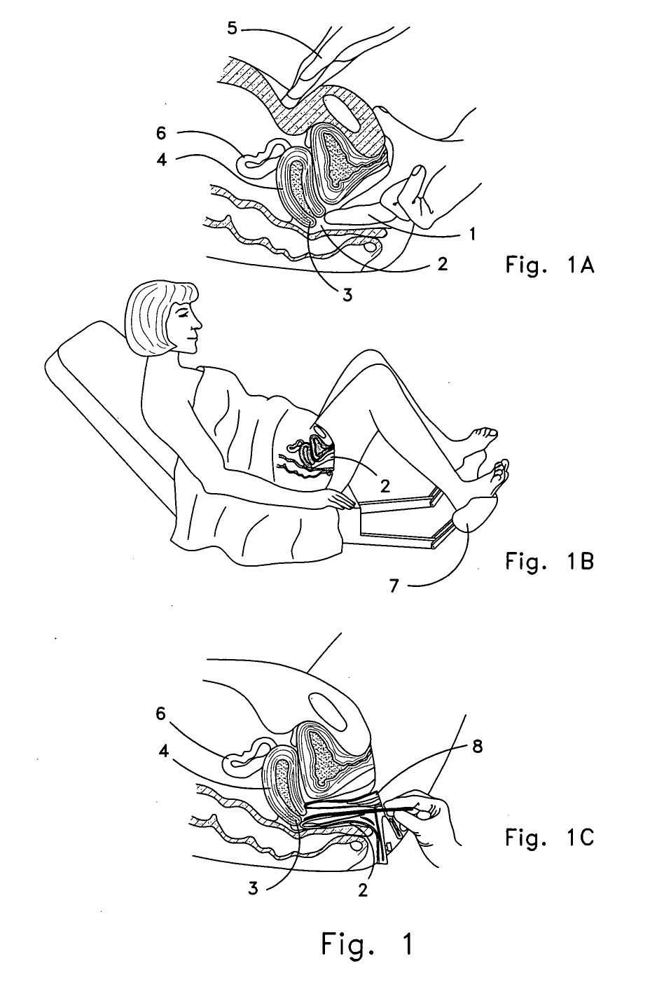

Referring now to the drawings in detail, FIG. 1 pertains to the prior art procedures. It shows a woman having the so-called pelvic exam or bimanual pelvic exam and Pap smear. This Figure helps to understand why women generally dislike the experience, which leads to the major problem of the Pap smear screen, namely the inability to persuade all women to undergo these check ups, and do so regularly.

In FIG. 1A, the clinician inserts a finger 1 into the woman's vagina 2 in order to touch the cervix 3 and stabilize the uterus 4 from the inside, while gently pressing down on the abdomen with his other hand 5, to palpate the uterus 4 and ovaries 6 from the outside. In FIG. 1B, the woman is shown in the recumbent position on the gynecological table with her legs apart, using the so-called stirrups 7. In FIG. 1C, the speculum 8 (a two-bladed cylindrical instrument made of stainless steel or plastic) is used to view and access the cervix 3 for taking a smear for the Pap smear test. This procedure of the prior art must be repeated several times over the span of many months, approximately somewhat over a year. Colposcopy with cervical biopsy is similar except that the colposcope (basically a speculum made into a microscope) affords a magnified view of the tissues, and the biopsy involves nipping off much more of the tissues than the smear, making it not just unpleasant but outright painful to the woman.

FIG. 2 is a perspective view of one preferred embodiment of the apparatus of the present invention. This affords a method of performing a quick and easy daily self-check in vivo, while actually monitoring a non-threatening variable such as particularly the woman's fertility status, i.e., whether she is within or outside of her brief fertile period near ovulation. The device is similar to a sanitary tampon dispenser with a small electronic control panel.

FIG. 3 is in two parts, 3A and 3B, and represents the probe of FIG. 2 inserted in the vagina, with two different orientations of the active elements of the probe. In FIG. 3A, the shown active element is not incontact with the cervix. FIG. 3B is a close-up perspective view of the probe component showing the placement of the probe in such an orientation that the active elements are in contact with a well-defined predetermined aspect of the epithelial tissues in the vagina, in this case one in contact with the cervix. It is to be understood that these are merely examples and that the probe may carry several active elements, and may perform more than one type of measurement via the multiplicity of active elements.

FIG. 3 shows the anatomical arrangement of the reproductive organs of a standing woman in a side view. FIGS. 3A and 3B show the posterior fornix region 31 at the upper end of the vaginal canal 32 into which extends the semispherical protrusion of the cervix 33. FIG. 3A depicts additional details of the anatomical arrangement of the reproductive system, including the body of the uterus 35 (from which the cervix 33 protrudes downward into the vagina 32), and including also the left and right ovaries 36 and 37 and left and right oviducts (Fallopian tubes) 38 and 39 that grow from the respective sides of the uterus 35, curving around the ovaries and ending in the funnel-like arrangements of the fimbria 310 and 311. The purpose of the fimbria 310 and 311 is to capture the egg when it ovulates by bursting out of one or the other ovary 36 or 37 at the time of ovulation to travel through the respective oviduct toward the uterus. (Fertilization would occur in the oviduct if sperm have been injected and traveled effectively through the cervix and uterus.) The probe 34 is inserted in the same manner as a vaginal tampon, and will naturally go as far as the posterior fornix region 31, at the far upper end of the vaginal canal 32, in the neighborhood of the protruding cervix 33.

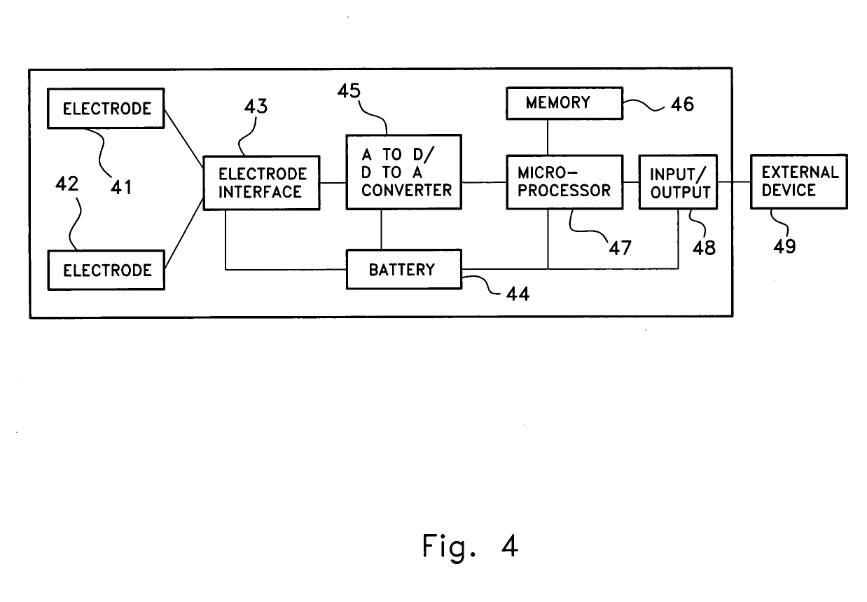

FIG. 4 is a block diagram of the basic configuration of the electronic module. Microprocessor 47 generates the waveform used in the probe measurement. The waveform is converted into an analog signal in converter 45 and is applied to the active elements 41 and 42 via the interface conditioning electronics 43. The response of the active elements is similarly converted into digital data by the converter 45 for processing by microprocessor 47. The processed data is displyed on LCD or LED display (not shown) and stored in memory 46 for optional downloading at a later date via input/output interface 48 to external device 49, which can be, for example, a computer.

The microprocessor 47 also performs processing of the measurement data. The routine for data evaluation can be described in terms of events and responses as follows.

Event 1:

Instant data (i.e., the current data point) fits the normal pattern of the probe cyclic profile, or fits the normal pattern AND the aberration pattern but the aberration incidence counter is below the preset value of repetitions (e.g., less than four consecutive cycles).

Response 1:

Normal result display routine displays the diagnostic interpretation that is the primary purpose of the device; and stores the raw data in memory.

Event 2:

Instant data fits the aberrant patter, and the aberration incidence counter is at the preset value of repetitions that triggers the prompt.

Response 2:

Warning routine is activated to display the prompt for the user to see a physician (with a view to undergoing the definitive examination by colposcopy with tissue biopsy). Raw data is stored in memory.

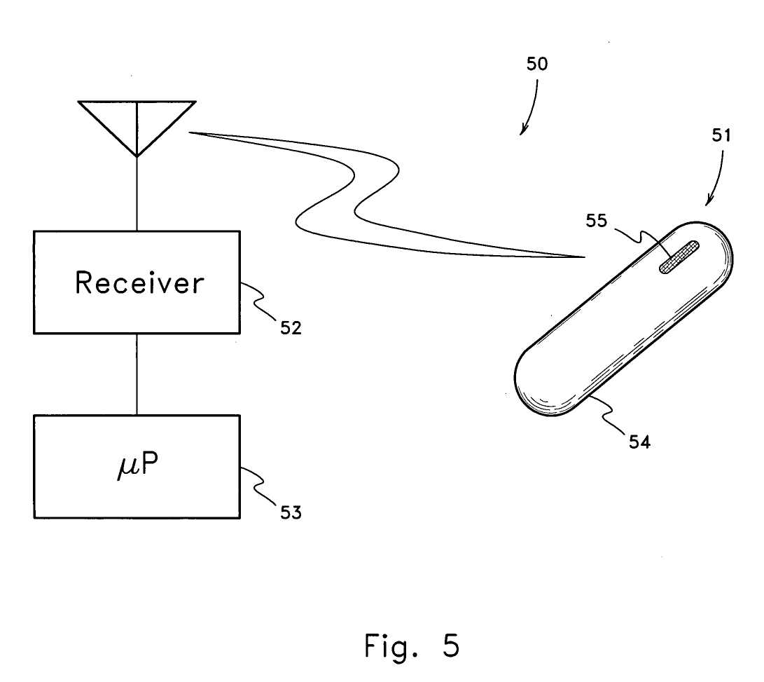

As shown in FIG. 5, the telemetric embodiment 50 of the present invention includes a transmitting vaginal insert 51, receiver 52, and microprocessor system 53. Transmitting vaginal insert 51 has a housing 54, to which at least one active element 55 is mounted. Active element 55 contacts the tissue of which the value of a physical parameter, indicative of the phase of the menstrual cycle or of the tissue's hormonal or physiological status, is measured. Transmitting vaginal insert 51 transmits this information to receiver 52. Receiver 52 reconstructs the transmitted signal and relays it to microprocessor system 53, where the information is processed.

The microprocessor system performs steps whereby the transmitted data is decoded, analyzed, stored, and displayed for the purpose of both the primary diagnostic indication and the secondary indication, the latter being the detection of cervical cancer-type aberration, resulting in a display indication such as “go to doctor”. This can only occur if the embedded program detects a consistently repeating aberration within or outside of the pattern expected for the primary diagnostic purpose of the daily self-check. Under normal circumstances, the display indicates, for example, INFERTILE DAY or FERTILE DAY 3, etc., as the case may be.

In one example of a specific embodiment of said electronic module the experiments involved admittance measurements with electrodes stimulated by a sinusoidal potential difference of between 10 mV and 500 mV peak to peak and a frequency between 1 kHz and 1000 kHz.

In FIG. 6 we illustrate the important point, namely that the housing 54 of the transmitting vaginal insert embodiment of the probe component may have any of a number of physical shapes such as, for example, a capsule (bolus) or a ring or a cylinder. These are merely examples of different solutions to the physical shape aspect of the telemetric probe, which pertains to the need to make and maintain a good, innocuous and reproducible tissue contact. The shown examples indicate that there is a a way to achieve reproducibility and non-irritability by altering the shape of the telemetric probe component. Such an alteration of the shape may include, e.g., additional features such as a more “anatomically ergonomic” shape, or such as an added “ear” as shown in 64, or a conical shape of a cylindrical insert as shown in 65.

FIG. 7 illustrates the insertion of one type of the vaginal insert embodiments of the probe, namely the ring with an added ear. Unlike with the main single-piece embodiment, this insertion is not performed every day because this version of the device is worn for as long as comfortable and needed, preferably over extended periods of time. The insertion is similar to that of a diaphragm, and the added ear affords two important aspects of the disclosed apparatus and method. Those are: 1. an improved means of handling the vaginal insert, and 2. securing a reproducible positioning of the probe in the vagina, in order to achieve reproducibility of measurement location from one act of measurement to the next.

Connections

The probe is connected to the electronic module in one of a number of conceivable ways, depending on whether the probe component is physically joined with the electronic module or not. The alternative ways of these connections are illustrated in FIGS. 2 and 5. The probe component may be connected with the electronic module either by wires or by any of a number of wireless means, such as by radio waves or by infrared radiation. The wires are generally embedded within the plastic body of the single-piece apparatus or within the plastic sheath of the cabling of a two-piece corded system. The wireless connection may be employed either within the single-piece embodiment or, more likely, in the telemetric embodiment. In either the wired or the wireless case, the purpose is to actuate the active elements under the control of the electronic module, and to receive and process the response to the actuation, i.e., the measurement result, within the electronic module. The result is displayed on the LCD or LED display of the electronic module, and the data is stored in memory included within the electronic module. The electronic module is further equipped with a means to optionally download the stored data to a peripheral device, upon demand. The said display provides the user the routinely measured diagnostic information, such as her fertility status, and only in the exceptional case of an electronically detected aberration it also provides a prompt for the woman-user to see a physician who will then have the option to download the stored data from the memopry of the electronic module.

Operation

When operating the apparatus of the present application, the user decides to take her daily vaginal reading in order to obtain her fertility status, or one of the other diagnostic readings such as whether pre-ovulatory or post-ovulatory for PMS management. The user is not specifically looking for an indication of pathology although she is aware that, if appropriate, the apparatus of the present invention will issue a cervical cancer related alert, prompting her to see a gynecologist. In a typical application of the apparatus, the woman user wishes to ascertain whether today is one of her very few fertile days whereby she could conceive a baby.

In accordance with 35 U.S.C. 112, the “best mode” of using the invention is as follows, using the main single-piece embodiment of the invention, the user makes her daily insertion of the probe as if it were a sanitary tampon (inserting as far as it goes). The user allows a few seconds, as required by the operating instructions, and, following a gentle audio indication that the electronic module has performed a steady-state reading or measurement, the user then withdraws the probe and reads the outcome on the display of the electronic module.

The functional relationship between the probe and the electronic module, the two main components, involve the actuation of the active elements embedded within the probe component and thus performing a measurement under the control of the electronic module main component. All data processing is then carried out within the electronic module, in the microprocessor or ASIC part of the microelectronic circuitry of the electronic module.

The display part of the electronic module will normally indicate the result of the measurement of the health condition as evaluated by the embedded electronics, and this will be typically the end of the user's daily routine. No unnecessary anguish of discomfort is caused by the procedure, which is normally concerned with self-monitoring of the woman user's fertility status or of her hormonal status such as in the peri-menopausal woman, or with yet other aspects of women's health (e.g., for PMS management the need is to differentiate the luteal phase from the follicular phase, i.e., post-ovulatory from pre-ovulatory).

Only under exceptional circumstances should the said “smart” electronics have reached a threshold number of consistently repeating aberrant patterns that are consistent with a predetermined pattern symptomatic of the pre-cancerous state, will the display so inform thee user, such as by indicating “go to doctor”. In this case, the user instructions will explain in more detail that the medical specialist will have available several months of data, stored in the memory. The data is provided for individual scrutiny and comparison with or against standard and aberrant patterns, and for physician-confirmed diagnosis that does warrant colposcopy and, tissue biopsy.

The “best mode” procedure with the alternative telemetric embodiment of the present invention is very similar. It only differs in that the insertion is not a daily routine (because the probe component is permanently worn in the vagina). Therefore, keeping herself still to allow for a steady-state measurement the user “wakes up” the vaginal insert with her detached electronic module. She does this also on a daily basis and for the same normal purpose of monitoring her fertility status or other unthreatehing condition. Everything else is as described for the* main single-piece embodiment of the probe.

The functional relationship between the two main components, i.e., the probe and the electronic module, is the same and only the means of communication between the two main components are different. In the single-piece embodiment, the communication between the two main components occurs within the single-piece, (or corded two-piece) structure such as via wires embedded within the structure, whereas in the alternative telemetric embodiment the communication between the disjointed main components is via electromagnetic waves such as radio waves. The end-result for the user is the same namely to view the diagnostic indication on the display part of the electronic module.

The functional relationship between the two main components involves the electronic module actuating the active elements of the probe and receiving the instant measurement data from the probe. All other electronic actions occur within the electronic module. They include storing the, instant data in memory and performing an electronic evaluation of the data. The evaluation is twofold, namely to assess the fertility status by fitting the data into a pattern representing the norm, such as the menstrual cyclic pattern, and report the outcome as, e.g., either INFERTILE or FERTILE DAY x (x=1, 2, . . . ).

The data evaluation involves also looking for whether an aberrant pattern, if obtained, has been obtained before, and if so, whether it fits a stored standard, aberrant pattern and/or whether the number of its consistent repetitions has reached a preset threshold (such as 3 or 6, as appropriate). If so, the electronic, module, issues a prompt to the, user via the alphanumeric display. Importantly, the secondary diagnostic evaluation, which is the main purpose of the disclosed method and apparatus, may be performed on the same measurement data as the primary diagnostics evaluation, or, it may involve a separate measurement and/or even separate active elements dedicated to the detection of the cervical cancer-indicative aberration.

1. A method of screening for early signs of cervical cancer comprising the following two fundamental steps:
i. the user performs daily or almost daily a diagnostic self-check for some other aspect of women's reproductive health, and
ii. the electronic testing for cervical cancer type tissue aberration is performed automatically in the background, invisible to the user, who only receives a prompt to see a physician if or when a preset condition of reproducibility is reached in the background evaluation of the measurement data.

2. The method of claim 1 wherein said aspect of women's health is chosen from the group consisting fertility status determination, differentiating between the luteal and follicular phases of the menstrual cycle such as for premenstrual syndrome management purposes, anticipating and/or detecting ovulation, assessing the hormonal status of vaginal tissues such as for hormone replacement therapy purposes, and monitoring the physiological status of vaginal tissues for any reason other than cervical cancer.

3. The method of claim 2 using a feminine self-testing device, and comprising the following steps:
i. placing in the vagina a probe having at least one or more active, elements for tissue evaluation;
ii. electronically stimulating, the active elements whereby performing the instant measurement of at least one parameter chosen from the group consisting of the phase of the reproductive cycle, the physiological status, and the hormonal status of the epithelium;
iii. storing the instant value of measurement data in memory;
iv. comparing said value with at least one reference value, i.e., evaluating the instant data in terms of the primary purpose such as fertility status determination, by applying a program that essentially looks for a fit of the instant data point into a standard menstrual cyclic pattern; and/or
v. comparing said value with at least one other reference value, i.e., evaluating the instant data in terms of the secondary purpose, by applying another data examination program; and
vi. displaying the outcome of step v. and/or of step vi. on the digital display of the electronic module.

4. The method of claim 3 wherein said probe is positioned so that at least one of said active elements contacts an anatomical feature chosen from the group consisting of the cervix and the vaginal fornix epithelium.

5. The method of claim 3 wherein said parameter is measured with at least one active element chosen from the group consisting of: electrode, light emitting diode, radio frequency emitter, high frequency ultrasound emitter, or emitter of any other form of energy which would be attenuated based upon the condition of the epithelial tissue.

6. The method of claim 3 wherein said diagnostic self-check is performed by applying more than one of the measurement methods with the active elements chosen from the group consisting of: electrode, light emitting diode, radio frequency emitter, high frequency ultrasound emitter, or emitter of any other form of energy which would be attenuated based upon the condition of the epithelial tissue.

7. The method of claim 3 wherein the primary purpose of the diagnostic self-check is the cervical cancer warning per se, i.e., by omitting step iv. in claim 3.

8. The method of claim 1 wherein the outcome of instant diagnostic measurement, and optionally all the stored raw data, is communicated to an external device for examination by a specialist, and the communication is by any of a conceivable electronic means such as are involved in corded or cordless telephony and conceivably over a network, private or public such as the Internet.

9. The method of claim 1 wherein said preset condition of reproducibility is that the aberrant cyclic pattern has been detected consecutively in a preset number of menstrual cycles, e.g., in the fourth or fifth consecutive menstrual cycle, at which time the user is prompted to see a physician with a view to undergoing a definitive diagnostic examination such as colposcopy with tissue biopsy.

10. An apparatus for the screening for early sighs of cervical cancer, which has the following attributes:
the screening involves the user performing daily or almost daily a diagnostic self-check for some other aspect of health,
the electronic testing for cervical cancer type of tissue aberration is performed automatically in the background, invisible to the user, and
the user is alerted to the need to see a physician only when and if a preset condition of reproducibility is reached in the background evaluation of the measurement data.

11. The apparatus of claim 10 wherein the apparatus includes a probe and an electronic module for performing cervical cancer bioassay in vivo, said probe and electronic module comprising:
a—means for introducing the active elements into the vagina, and
b—means for the measurement with the active elements of at least one physical variable under the control of the electronic module, and evaluating the data within said module.

12. The probe of claim 1-1 wherein the probe is physically joined with the electronic module in a single-piece or in a corded structure.

13. The probe of claim 11 wherein the probe is not physically joined with the electronic module and is in the form of a permanently or non-permanently worn vaginal insert, which communicates telemetrically with said electronic module.

14. An electronic module for performing cervical cancer bioassay in vivo, said electronic module comprising:
means for the measurement with the probe's active elements of a physical variable that is characteristic of the hormonal and physiological variations in the tissues such as alternating current, admittance or any of their components, or other, derived or computed, parameter or any kind of electromagnetic energy, using for the active elements' stimulation any of a number of conceivable waveforms such as but not limited to at least one sine-wave,
means for the evaluation of instant measurement data,
means for the communication of the evaluation of the instant measurement results, and
means for downloading the stored raw data upon demand.

15. The electronic module of claim 14 wherein said means for the measurement involve admittance measurement with electrodes stimulated by a sinusoidal potential difference of between 10 mV and 500 mV peak to peak and frequency between 1 kHz and 1000 kHz.
